(19) United States
(12) Patent Application Publication
(10) Pub. No.: US 2020/0121927 A1
(43) Pub. Date: Apr. 23, 2020

(54) Cochlear Implant System with Microphone and Sound Processor on a Consumer Device

(71) Applicant: Thomas Hampton, East Longmeadow, MA (US)

(72) Inventor: Thomas Hampton, East Longmeadow, MA (US)

(21) Appl. No.: 16/164,770

(22) Filed: Oct. 18, 2018

(51) Int. Cl. A61N 1/36; A61N 1/05; A61N 1/378

(52) U.S. Cl. CPC A61N 1/36038; H04R 25/606; A61N 1/378; A61N 1/0541

(57) ABSTRACT

A cochlear implant system that includes a user's device and a cochlear implant. A microphone can either be in-built into the user's device (for example smartphone) or can be an external microphone (for example a wireless microphone connected to a smart-watch). The microphone collects sound signals and sends it to user's device for sound processing. After filtering the sound, the processed sound signals are sent to a radio frequency (for example Bluetooth) receiver located behind the ear and sends the signal through the skin through a coil coupled transcutaneous system to the implant in the user's ear, where an electrode array correspondingly generates acoustic stimulations in user's cochlea.

### BACKGROUND

Cochlear implant systems bypass hair cells located in the cochlea by presenting electrical stimulation directly to the auditory nerve fibers.

A cochlear implant system can have a behind-the-ear (BTE) processor or a body worn processor. Typically a BTE processor attaches over the user's ear through a hook that holds the BTE processor in place. A cable attaches the BTE processor to the implanted headpiece. The BTE processor suffers from space constraints and, therefore, power and processing capacity constraints exist.

Typically these limitations of power and processing capacity are overcome by using a body worn processor. The body worn processor does not have the size and weight constraints that are associated with a BTE processor. Therefore, power and processing capacity of the body worn processor can be significantly greater than BTE processors. Similar to the BTE processor, a cable attaches the body worn processor to the implanted headpiece.

However, the body worn processor requires a longer cable that is both difficult to manage and is not visually appealing. It also requires extra cost.

Therefore, there is a need for even higher power and processing capabilities for faster and better sound quality and convenience.

### BRIEF DESCRIPTION OF THE DRAWINGS

The accompanying drawings illustrate various embodiments of the principles described herein and are a part of the specification. The illustrated embodiments are merely examples and do not limit the scope of the claims.

FIG. 1 is a block diagram of a cochlear implant system, in accordance to various embodiments of the disclosure.

FIG. 2 is a block diagram of a cochlear implant system, in accordance to various embodiments of the disclosure.

Throughout the drawings, identical reference numbers designate similar, but not necessarily identical, elements.

### DETAILED DESCRIPTION

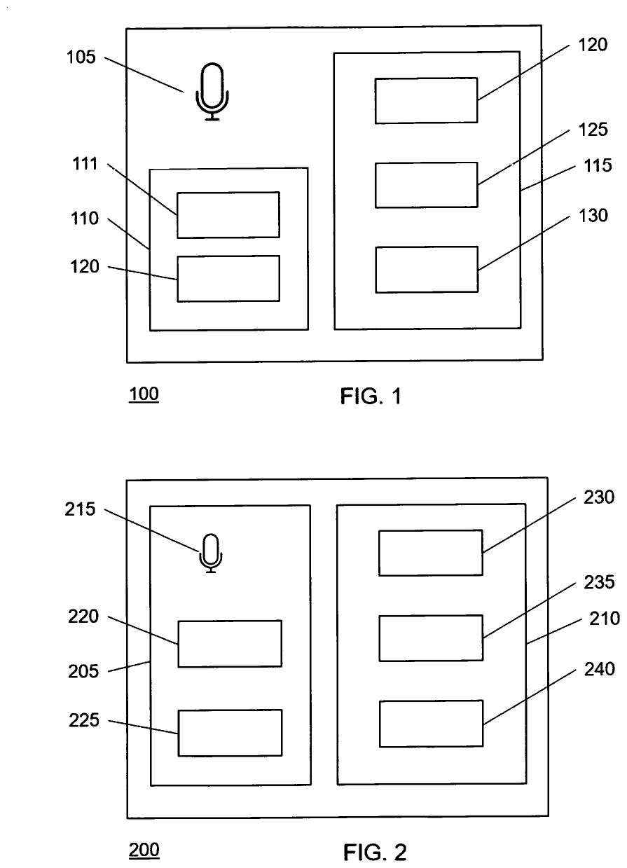

FIG. 1 is a block diagram of a cochlear implant system 100, in accordance to various embodiments of the present disclosure. The cochlear implant system 100 has a microphone 105, a user's device 110 and a cochlear implant 115. The microphone 105 receives a plurality of sound signals. The plurality of sound signals is a combination of a plurality of sound frequencies and/or a plurality of noise frequencies. In other words, the plurality of sound signals received in the microphone 105 has sound that is both desired and undesired by the user of the cochlear implant system 100. The cochlear implant system 100 further has the user's device 110. For example, the user's device 110 comprises a mobile phone, a watch, a tablet, a handheld device or a wearable consumer device, or a combination thereof. The user's device 110 has a speech processor 111 and a transmitter 120. The speech processor 111 isolates the plurality of speech frequencies from the plurality of sound signals received by the microphone 105. The speech processor 111 filters out the noise signal from the received sound signal. In accordance with various embodiments of the present disclosure, the speech processor 111 is enabled by an app or program on the user's device 110. In at least one embodiment of the present disclosure, a user can install an app (or software) on his handheld or wearable device that allows processing of sound signals collected by the microphone 105. In accordance with embodiments of various embodiments of the present invention, the microphone 105 can be a part of the user's device 110. The transmitter 120 then transmits filtered signals. In accordance with embodiments of various embodiments of the present invention, the transmitter 120 is a radio frequency antenna. Thereafter, the filtered speech signals, i.e., the plurality of speech frequencies, are received by a radio frequency transceiver 125 in the cochlear implant 115. An implant receiver 130 receives the plurality of speech frequencies from the radio frequency transceiver 125. In accordance with various embodiments of the present disclosure, the implant's receiver (which is externally worn) 130 receives the plurality of speech frequencies from the radio frequency transceiver 125 through a coil-coupled transcutaneous system. Moreover, an implant electrode array 135 creates a plurality of acoustic stimulation in the user's ears based on the plurality of frequencies received by the implant receiver 130. The plurality of acoustic stimulation corresponds to the plurality of desired frequencies. For example, if a user is using a mobile phone, an external microphone can collect sound signals transmitted to the mobile phone. An app can use the mobile phone's processing power to process the sound signals to isolate desired signals. These isolated speech signals can be transmitted to a receiver behind the user's ear. These can be transmitted to an implanted electrode array, which subsequently generates acoustic stimulations in the user's ear.

FIG. 2 is a block diagram a cochlear implant system 200, in accordance to various embodiments of the present disclosure. The cochlear implant system 200 has a user's device 205 and the cochlear implant 210. The user's device 205 comprises a mobile phone, a watch, a tablet, a handheld device, a wearable device, or a combination thereof. It will be understood to a person having ordinary skill in the art that the user's device 205 are mentioned for ease of understanding and does not intend limit the scope of the invention. The user's device 205 has a microphone 215, a speech processor 220 and a transmitter 225. The microphone 215 receives a plurality of sound signals that has a plurality of sound frequencies. For example, the microphone 215 may comprise an in-built microphone of a mobile phone. Thereafter, the speech processor 220 receives the plurality of sound signals from the microphone 215 and isolates the plurality of desired frequencies from the plurality of sound signals. The filtered desired frequencies are transmitted by the transmitter 225. The transmitter 225 is a radio frequency antenna. It will be understood to a person ordinarily skilled in the art that the transmitter 225 is described to a radio frequency antenna for the sake of clarity and in accordance with current state of technology. Possible variants of a radio frequency antenna can also be used. A radio frequency transceiver 230 receives the plurality of filtered frequencies from the transmitter 225 in the user's device 205. The cochlear implant 210 is inserted in the user's ear. It will be understood to a person ordinarily skilled in the art that the cochlear implant 210 is described to be surgically implanted in user's ear. An implant receiver 235 receives the plurality of speech frequencies from the radio frequency transceiver 230 through a coil-coupled transcutaneous system. Thereafter, an implant electrode array 240 creates a plurality of acoustic stimulation in the user's ear based on the plurality of speech frequencies received by the implant receiver 235. The plurality of acoustic stimulation corresponds to the plurality of speech frequencies. In accordance with various embodiments of the present disclosure, the speech processor 200 is enabled by an app on the user's device. For example, a user can download an app on his mobile phone. The app can use the mobile phone's processing power to process sound signals to isolate speech signals and filter out noise frequencies. In another example, if a user is using a mobile phone, the in-built microphone can collect sound signals and an app controlled by the user can use the mobile phone's processing power to process the sound signals to isolate a specific person's speech signals. These isolated speech signals can be transmitted to a receiver behind the user's ear. These can be transmitted to an implanted electrode array, which subsequently generates acoustic stimulations in the user's ear.

The preceding description has been presented only to illustrate and describe embodiments of the present disclosure. This description is not intended to be exhaustive or to limit the scope of the invention. Many modifications and variations are possible in light of the above teaching.

1. A cochlear implant system comprising:
a. a microphone configured to receive a plurality of sound signals, wherein the sound signals are defined by a plurality of sound frequencies and a plurality of noise frequencies;
b. a user's device comprising:
i. a speech processor configured to receive the plurality of sound signals from the microphone, further wherein the sound processor isolates a plurality of desired frequencies from the plurality of sound signals received from the microphone; and
ii. a transmitter configured to transmit the plurality of desired frequencies;
c. a cochlear implant capable of being inserted in a user's ear, wherein the cochlear implant comprises:
i. a radio frequency transceiver configured to receive the plurality of desired frequencies from the transmitter in the user's device;
ii. an implant receiver configured to receive the plurality of desired frequencies from the radio frequency transceiver; and
iii. an implant electrode array configured to create a plurality of acoustic stimulation in user's ear based on the plurality of desired frequencies received by the implant receiver, wherein the plurality of acoustic stimulation corresponds to the plurality of desired frequencies.

2. The cochlear implant system as recited in claim 1, wherein the user's device comprises a microprocessor controlled personal device further comprising a mobile phone, a watch, a tablet, a handheld device, a wearable device, or a combination thereof.

3. The cochlear implant system as recited in claim 1, wherein the speech processor is enabled by an app running on the user's device.

4. The cochlear implant system as recited in claim 1, wherein the microphone is integrated with the user's device.

5. The cochlear implant system as recited in claim 1, wherein the radio frequency transmitter of the user's device is a radio frequency receiver.

6. The cochlear implant system as recited in claim 1, wherein the implant receiver is configured to receive the plurality of desired frequencies from the radio frequency transceiver through a coil-coupled transcutaneous system.

7. A cochlear implant system comprising:
a. a microphone configured to receive a plurality of sound signals, wherein the plurality of sound signals is defined by a plurality of sound frequencies and a plurality of noise frequencies;
b. a user's device comprising:
i. a speech processor configured to receive the plurality of sound signals from the microphone, further wherein the sound processor isolates the plurality of speech frequencies from the plurality of sound signals; and
ii. a transmitter configured to transmit the plurality of speech frequencies; and
c. a cochlear implant capable of being inserted in user's ear, wherein the cochlear implant comprises:
i. a radio frequency transceiver configured to receive the plurality of speech frequencies from the transmitter in the user's device;
ii. an implant receiver configured to receive the plurality of speech frequencies from the radio frequency transceiver, wherein the implant receiver is configured to receive the plurality of speech frequencies from the radio frequency transceiver through a coil-coupled transcutaneous system; and
iii. an implant electrode array configured to create a plurality of acoustic stimulation in user's ear based on the plurality of desired frequencies received by the implant receiver, wherein the plurality of acoustic stimulation corresponds to the plurality of speech frequencies.

8. The cochlear implant system as recited in claim 7, wherein the user's device comprises a microprocessor controlled personal device, said microprocessor controlled personal device further comprising a mobile phone, a watch, a tablet, a handheld device, a wearable device, or a combination thereof.

9. The cochlear implant system as recited in claim 7, wherein the speech processor is enabled by an app running on the user's device.

10. The cochlear implant system as recited in claim 7, wherein the microphone is integrated with the user's device.

11. The cochlear implant system as recited in claim 7, wherein the transmitter of the user's device is a radio frequency receiver.

12. A cochlear implant system comprising:
a. a user's device, wherein the user's device is one (or a combination) of a mobile phone, a watch, a tablet, a handheld device and a wearable device, wherein the user's devices comprising:
i. a microphone configured to receive a plurality of sound signals, wherein the plurality of sound signals is defined by a plurality of sound frequencies and a plurality of noise frequencies;
ii. a speech processor configured to receive the plurality of sound signals from the microphone, further wherein the sound processor isolates the plurality of desired frequencies from the plurality of sound signals; and
iii. a transmitter configured to transmit the plurality of desired frequencies, wherein the transmitter is a radio frequency antenna; and
b. a cochlear implant capable of being inserted in user's ear, wherein the cochlear implant comprising:
i. a radio frequency transceiver configured to receive the plurality of speech frequencies from the transmitter in the user's device;
ii. an implant receiver configured to receive the plurality of speech frequencies from the radio frequency transceiver, wherein the implant receiver is configured to receive the plurality of desired frequencies from the radio frequency transceiver through a coil-coupled transcutaneous system; and
iii. an implant electrode array configured to create a plurality of acoustic stimulation in user's ear based on the plurality of speech frequencies received by the implant receiver, wherein the plurality of acoustic stimulation corresponds to the plurality of desired frequencies.

13. The cochlear implant system as recited in claim 12, wherein the speech processor is enabled by an app on the user's device.

14. The cochlear implant system as recited in claim 13, wherein the microphone is integrated with the user's device.
